(19) United States
(12) Patent Application Publication
(10) Pub. No.: US 2003/0143704 A1
(43) Pub. Date: Jul. 31, 2003

(54) Method to separate ethanol fermented from sugar derived from a biomass

(76) Inventor: Gene E. Lightner, Federal Way, WA (US)

Correspondence Address: Gene E. Lightner, 706 S.W. 296th St., Federal Way, WA, 98023, US

(21) Appl. No.: 09/912,893

(22) Filed: Jul. 25, 2001

(51) Int. Cl. C12P 7/06; C07C 27/26; C07C 29/74

(52) U.S. Cl. 435/161; 568/913

(57) ABSTRACT

This is a method to remove ethanol from fermentation of sugars contained in a vessel. By providing carbon dioxide, containing ethanol, to the vessel, ethanol and volatile compounds formed by fermentation are humidified by the carbon dioxide. A supply of sugars for fermentation within the vessel is employed to form ethanol and carbon dioxide. Upon combining carbon dioxide, produced by fermentation, with the carbon dioxide, containing ethanol, ethanol humidified carbon dioxide is formed. Resulting humidified carbon dioxide is removed from the fermentation vessel and subjected to means for condensing ethanol from humidified carbon dioxide to supply carbon dioxide, containing ethanol, for recycle, and purged carbon dioxide, containing ethanol, to remove carbon dioxide produced by fermentation. Fermentation broth is removed from the vessel to maintain vessel level. Thereby ethanol is condensed from the humidified carbon dioxide to produce ethanol and carbon dioxide, containing ethanol, for recycle and removing carbon dioxide produced by fermentation and maintaining broth level within the fermentation vessel.

### BACKGROUND OF THE INVENTION

Present day interest in hydrolysis of biomass is to provide an alternative fuel source to avoid dependence on unreliable imported petroleum crude oil for liquid fuels. Characteristic dry biomass composition is: lignin 25%, hemicellulose 25%, amorphous cellulose 10%, and crystalline cellulose 40%.

A method of removal of hemicellulose derived from a biomass is described by Grothmann, et al, in U.S. Pat. No. 5,125,977, wherein two stages are employed for hydrolysis relying on dilute acid to remove hemicellulose and form water soluble five and six carbon sugars

“Recombinant yeasts for effective fermentation of glucose and xylose” is the title of a process presented within U.S. Pat. No. 5,789,210, by Ho, et al, wherein a mixture of water soluble five and six carbon sugars can be subjected to fermentation to form ethanol.

A method for extraction of ethanol from fermentation is disclosed within U.S. Pat. No. 4,517,298, by Tedder, depends on an organic solvent to extract ethanol from an insoluble fermentation broth. Accordingly, the organic solvent extracate containing dissolved ethanol is subjected to distillation to be separated from ethanol and provide a raffinate of organic solvent.

A basic object of this invention is to remove ethanol carbon dioxide and volatile compounds created by fermentation within a fermentation vessel.

An essential object of this invention is to add carbon dioxide, containing ethanol, to the fermentation vessel to humidify ethanol and volatile compounds and remove humidified carbon dioxide from the fermentation vessel.

A distinct object of this invention is to prevent concentration of ethanol within the fermentation process from reaching a concentration of ethanol lethal to yeasts and enzymes within the fermentation vessel.

A particular object of this invention is means for removing ethanol from humidified carbon dioxide, wherein the humidified carbon dioxide forms condensed ethanol, which may require use of a demister, to remove ethanol separated from carbon dioxide containing ethanol.

An additional object of this invention is to utilize a hydrolyzate containing water soluble sugars and a dilute acid to supply water soluble sugars for fermentation.

Still another object of this invention is to retain sugars within the fermentation vessel. With the above and other objects in view, this invention relates to the novel features and alternatives and combinations presently described in the brief description of the invention.

### PRINCIPLES APPLIED BY THE INVENTION

The principles applied herein employ Dalton's law and Raoult's law. Dalton's law of partial pressure may be expressed mathematically as P=p_{A}+p_{B} where p_{A} and p_{B} are the partial pressures of vapors A and B respectively and P is the total pressure described by F. Daniels, Outlines of Physical Chemistry, page 198. For only A and B, P=p_{A}+p_{B}, and the mole ratio of B to A is p_{B}/p_{A}=p_{B}/P−p_{B}.

The weight ratio of A/B is p_{B}/P−p_{B} (molecular weight of B)/(average molecular weight of P−p_{B}). This is the equation used for humidity calculations when A is a gas and B is the vapor humidified described by G. G. Brown et al., Unit Operations, page 542.

Raoult's law of partial pressure may be expressed mathematically as pₛₒₗᵥₑₙₜ=p^{o}ₛₒₗᵥₑₙₜ×Nₛₒₗᵥₑₙₜ where pₛₒₗᵥₑₙₜ is the partial vapor pressure of the solvent, p^{o}ₛₒₗᵥₑₙₜ is the vapor pressure of the solvent times the mole fraction, N, of the solvent in a solution described by F. Daniels op. cit., page 202. Applying Raoult's law, let N=0.1 (the mole fraction of ethanol in a fermentation broth) and p^{o}ₑₜₕₐₙₒₗ at a temperature of 100° F. the partial vapor pressure=2.5 psia, then pₑₜₕₐₙₒₗ=0.1×2.5 psia=0.25 psia.

The molecular weight of ethanol=46 and carbon dioxide has a molecular weight=44.

Applying the equation used for humidity, and making P=15 psia for a total pressure of humidified carbon dioxide, the weight ratio of ethanol carbon dioxide is 0.25/15−0.25×46/44=0.018 lb. of ethanol/lb. of carbon dioxide.

Thus a fermentation process can have ethanol removed by co-mingling carbon dioxide with the fermentation process to form carbon dioxide humidified with ethanol. The carbon dioxide, humidified with ethanol, can also contain other humidified compounds.

Raoult's law predicts that any volatile compound will form a partial vapor pressure of the volatile compound depending or the vapor pressure and mole fraction of the volatile compound in the fermentation process. The equation used for humidity asserts that when a gas is humidified, the humidified gas may contain any partial vapor pressure of a volatile compound. Thus, if the humidified carbon dioxide contains a partial vapor pressure of a volatile compound contained in the fermentation broth of the same partial vapor pressure of the same volatile compound then further humidification of the volatile compound will not occur.

The same temperature of fermentation broth and the humidified carbon dioxide is assumed. For additional information, review F. Daniels, Outlines of Physical Chemistry and G. G. Brown, et al., Unit Operations.

A means of removal of hemicelhlulose from a biomass is disclosed by Grothmann, et al, in U.S. Pat. No. 5,125,977, wherein two stages are employed, relying on dilute acid, to hydrolyze hemicellulose contained within a biomass to produce a hydrolyzate containing xylose.

Thus two individual stages provide relative ease and relative difficulty of hemicellulose hydrolysis to prevent or limit formation of furfural. Accordingly a hydrolyzate containing xylose is created by hydrolysis of hemicellulose within a biomass employing a dilute acid.

Fermentation of sugars is best carried out at a temperature range of 27° C. to about 35° C. and a pH range of 3.0 to 5.0 as described by Hunt in The Gasohol Handbook, page 87. Nutrition requirements to promote cell growth are the chemical elements such as carbon, nitrogen and phosphorus nutrients used for fermentation as contributed by Hunt, op. cit., page 88. Two basic concerns that govern the activity of yeast cells are; excessively high sugar concentrations can inhibit the growth of yeast cells and concentration of ethanol of about 12% is lethal to yeast cells as disclosed by Hunt op. cit., pages 88-89. Sugars utilized for fermentation must be sterile to prevent growth of unwanted microbial contaminants as disclosed by Hunt, op. cit., pages 89-90. Cooling towers relying on air for cooling water is described by Brown, op. cit., pages 552-553.

### BRIEF DESCRIPTION OF THE INVENTION

The present invention in its broadest aspect, comprises a method to remove ethanol from fermentation broth contained in a vessel. The preferred method employs carbon dioxide, supplied to the fermentation vessel, to humidify ethanol and volatile compounds formed by fermentation. By providing a supply of sugars to the fermentation vessel, ethanol and carbon dioxide are formed from fermentation. Carbon dioxide, containing ethanol, is provided to the vessel to humidify ethanol within the fermentation vessel. By separating the resulting humidified carbon dioxide, containing carbon dioxide formed from fermentation, from the fermentation vessel, and removing means for removing ethanol from the humidified carbon dioxide to supply carbon dioxide, containing ethanol, for recycle, and purged carbon dioxide, containing ethanol, to remove carbon dioxide produced by fermentation. Level of broth within the fermentation vessel is maintained by removal of broth. The removed fermentation broth is subjected to distillation to produce ethanol vapor and a raffinate substantially devoid of ethanol. Purged carbon dioxide, containing ethanol, is absorbed by said raffinate to produce carbon dioxide substantially devoid of ethanol and a solution containing ethanol. The solution containing ethanol, with the broth, is subjected to distillation to produce ethanol vapor and a raffinate substantially devoid of ethanol. Ethanol vapor is combined with humidified carbon dioxide. Thereby products of fermentation are removed, and the fermentation vessel level is maintained by removal of broth.

Characteristics of the invention include;

Sugars to form ethanol and carbon dioxide, from fermentation broth, create ethanol and volatile components.

Carbon dioxide, containing ethanol, is provided to the vessel to humidify ethanol within the fermentation vessel.

Humidified carbon dioxide, containing ethanol and other volatile components from fermentation, is parted from the fermentation vessel.

Humidified carbon dioxide produces condensed ethanol and a supply of carbon dioxide, containing ethanol, for recycle.

Broth level within the fermentation vessel is removed to maintain level of broth within the fermentation vessel.

Temperature is maintained within the fermentation both at substantially isothermal conditions and pH level is likewise maintained.

### BRIEF DESCRIPTION OF THE DRAWINGS

The features that are considered characteristic of this invention are set forth in the appended claims. This invention, however, both as to its origination and method of operations as well as additional advantages will best be understood from the following description when read in conjunction with the accompanying drawings in which:

FIG. 1 is a flow sheet denoting the invention as set forth in the appended claims.

FIG. 2 is a flow sheet denoting a method to condense ethanol employing water.

FIG. 3 is a flow sheet denoting a method to produce a hydrolyzate from a biomass.

### DETAILED DESCRIPTION OF THE PREFERRED EMBODIMENT

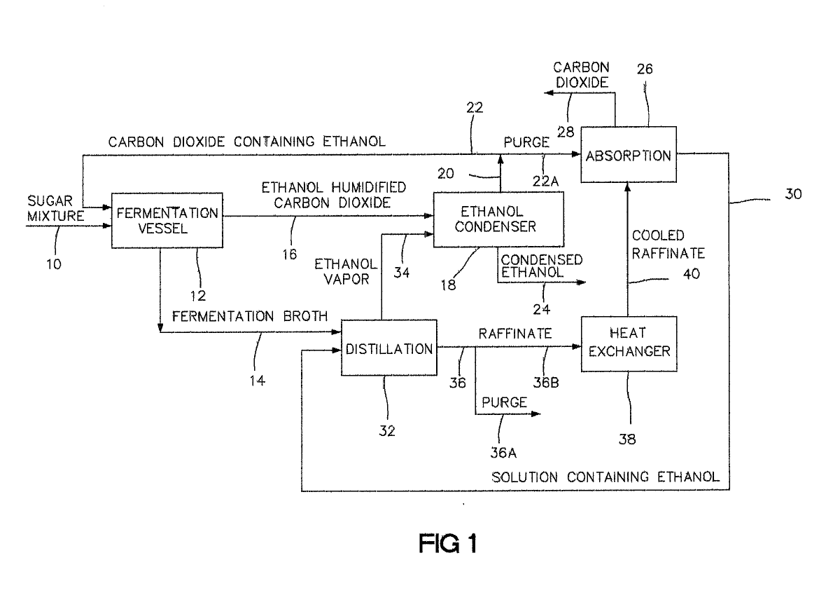

In the preferred embodiment of the present invention, carbon dioxide, containing ethanol, is added to a fermentation vessel, to humidify the carbon dioxide with ethanol within fermentation broth. The predetermined operating level of temperature range within fermentation is about 27° C. to about 35° C. The flow diagram of FIG. 1 illustrates the general preferred embodiment of the present invention. In the diagram, rectangles represent stages, operations or functions of the present invention and not necessarily separate components. Arrows indicate direction of flow of material in the method.

Referring to FIG. 1, sugar mixture 10 is conveyed to a fermentation vessel 12 and subjected to fermentation to form carbon dioxide and ethanol. Carbon dioxide, containing ethanol, is added within the vessel 22. The ethanol humidified carbon dioxide combined with carbon dioxide formed by fermentation, is separated from the fermentation vessel 12 to form ethanol humidified carbon dioxide 16 which is conveyed to ethanol condenser stage 18 to generate condensed ethanol 24 and form carbon dioxide, containing ethanol, 20 which is purged to produce purge 22A and produce carbon dioxide, containing ethanol, 22 for recycle to fermentation vessel 12. The purge 22A is conveyed to ethanol absorption stage 26 and subjected to absorption from cooled raffinate 40, cooled by heat exchanger stage 38. Absorption stage 26 creates carbon dioxide substantially devoid of ethanol 28 and produces a solution containing ethanol 30. The solution containing ethanol 30 is conveyed to distillation stage 32 to create ethanol vapor 34 transferred to ethanol condenser stage 18 to generate condensed ethanol 24.

Distillation stage 32 upon vaporization of ethanol produces a raffinate substantially devoid of ethanol 36 divided into purge 36A and raffinate 36B. Raffinate 36B is conveyed to heat exchanger stage 38. to produce cooled raffinate 40.

Heat exchanger stage 38. is commonly supplied water from a cooling tower or a well. Fermentation broth 14, removed from fermentation vessel 12, is conveyed to distillation stage 32 to create ethanol vapor 34 transferred to ethanol condenser stage 18 for formation of condensed ethanol 24. Condenser stage 18 can be subjected to pressurization, or cooled to produce condensed ethanol. A singular distillation stage 32 accordingly substantially removes ethanol from fermentation broth 14 and a solution containing ethanol 30 to produce a raffinate 36 substantially devoid of ethanol. The method described in FIG. 1 employs humidification for transmitting ethanol from fermentation to insoluble carbon dioxide contained within carbon dioxide, containing ethanol, 22. Humidified carbon dioxide 16 may contain various volatile compounds from the fermentation. The method can be operated by continuous operation. Ethanol humidified carbon dioxide 16 can be pressurized to condense ethanol 24 from ethanol humidified carbon dioxide 16. Sugars, capable of fermentation to produce ethanol and carbon dioxide, consist of the group which include glucose and xylose including an individual or a combination thereof. Sugars utilized for fermentation must be sterile.

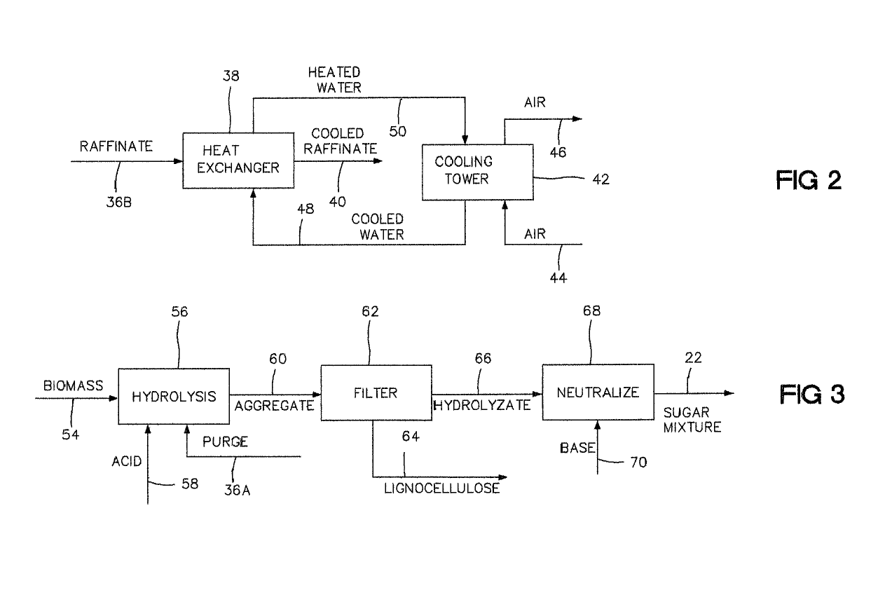

Referring to FIG. 2, After purging, raffinate 36A, is conveyed to heat exchanger stage 38 which is supplied by cooled water 48 from a cooling tower 42 utilizing ambient air 44 to cool water and create humidified air 46 for discharge. The heat exchanger stage 38 exchanges cooled water 48 to produce heated water 50 which is transmitted to cooing tower stage 42. The heat exchanger stage 38 produces cooled raffinate 40. The heat exchanger stage 38 function could be performed by water supplied from a well.

Referring to FIG. 3, biomass 54 is transported to hydrolysis stage 56, combined with acid 58, and with purge 36A to accomplish hydrolysis of hemicellulose, contained within biomass, 54. Aggregate of solids and sugars 60, formed by hydrolysis, is transported to filter stage 62. Filter stage 62 performs filtration to form lignocellulose 64 and hydrolyzate 66. Hemicellulose, contained in biomass, is accordingly hydrolyzed to produce lignocellulose 62 substantially devoid of hemicellulose. Hydrolyzate 66 is transported to neutralize stage 68, wherein the hydrolyzate is neutralized 66 to a pH of about 5 with base 70 and form a sugar mire 22. Acid within hydrolyzate 66 containing sugars is accordingly neutralized. Hemicellulose upon hydrolysis forms xyloses and glucose. Thus sugar mixture 22, containing glucose, is subjected to fermentation to form ethanol. Base 70 is often a hydroxide but can be calcium carbonate to neutralize acid.

What is claimed is:

1. A method to separate ethanol from a fermentation process contained in a vessel, which comprises:
providing a fermentation vessel in which ethanol and carbon dioxide are produced by fermentation, and
providing carbon dioxide, containing ethanol, to said vessel to humidify said ethanol within said vessel by said carbon dioxide, containing ethanol, and
providing a supply of sugars for fermentation to form ethanol and carbon dioxide, and
combining said carbon dioxide, produced by fermentation, with the carbon dioxide, containing ethanol, to form ethanol humidified carbon dioxide, and
separating the resulting humidified carbon dioxide from the fermentation vessel, and
removing means for condensing ethanol from said humidified carbon dioxide to supply carbon dioxide, containing ethanol, for recycle, and purged carbon dioxide containing ethanol to remove carbon dioxide produced by fermentation, and
removing fermentation broth from said vessel to maintain vessel level thereby removing ethanol from the humidified carbon dioxide to produce ethanol and carbon dioxide, containing ethanol, for recycle and removing carbon dioxide produced by fermentation and substantially maintaining broth level within the fermentation vessel.

2. The method of claim 1 wherein said fermentation takes place in which ethanol and carbon dioxide are produced from the group of sugars which include glucose and xylose including an individual or a combination thereof.

3. The method of claim 1 where said fermentation vessel is established and maintained at a predetermined ethanol level.

4. The method of claim 1 wherein said fermentation vessel is established at a predetermined temperature and maintained at substantially isothermal conditions.

5. The method of claim 1 wherein said fermentation broth, is established at a predetermined level of pH within said vessel and substantially maintained at the established pH level.

6. The method of claim 1 wherein said fermentation broth is established and maintained at a predetermined level of nutrients used for fermentation.

7. The method of claim 1 wherein said fermentation vessel is provided with sterile sugars to maintain fermentation.

8. The method of claim 1 wherein said removing means for condensing ethanol from said humidified carbon dioxide, said humidified carbon dioxide is subjected to an ethanol condenser, supplied by water, to produce condensed ethanol and carbon dioxide, containing ethanol, for recycle.

9. The method of claim 1 wherein said removing means for condensing ethanol from said humidified carbon dioxide, said humidified carbon dioxide is subjected to pressurization, to produce condensed ethanol and carbon dioxide, containing ethanol, for recycle.

9. The method of claim 1 wherein said broth is subjected to distillation to form ethanol vapor and a raffinate.

10. The method of claim 1 wherein the purged carbon dioxide, containing ethanol, is subjected to absorption by said raffinate, defined within claim 9, to provide a solution containing ethanol and removed carbon dioxide, produced by fermentation, substantially devoid of ethanol.

11. The method of claim 10 wherein the solution containing ethanol is subjected to distillation to produce an ethanol vapor and a raffinate.

12. The method of claim 11 wherein the distillation and, the distillation defined within claim 9, are combined to form a singular distillation.

13. The method of claim 9 wherein said raffinate and raffinate defined within claim 9, are combined and purged to maintain substantially constant broth level within the fermentation vessel.

14. The method of claim 13 wherein the purged raffinate is combined with acid to become dilute acid for hydrolysis to form a hydrolyzate.

15. The method of claim 1 wherein said sugars are obtained from a hydrolyzate derived from dilute acid hydrolysis of a biomass.

16. The method of claim 15 wherein the hydrolyzate is neutralized to form a sugar solution having a pH of about 5.

17. The method of claim 1 wherein said sugars are sterile.

18. The method of claim 1 wherein said method is continuous.

19. The method of claim 1 wherein said sugars are obtained from hydrolysis of a biomass including glucose.
